(19) United States
(12) Patent Application Publication
(10) Pub. No.: US 2011/0020785 A1
(43) Pub. Date: Jan. 27, 2011

(54) Diagnostic Information Generation and Use

(75) Inventors: Thomas J. Lowery, JR., Belmont, MA (US); Tuan A. Elstrom, Lake Bluff, IL (US); Douglas A. Levinson, Sherborn, MA (US); James J. Koziarz, Highland Park, IL (US)

Correspondence Address: STERNE, KESSLER, GOLDSTEIN & FOX P.L.L.C., 1100 NEW YORK AVENUE, N.W., WASHINGTON, DC, 20005, US

(73) Assignee: T2 Biosystems, Inc., Cambridge, MA (US)

(21) Appl. No.: 12/670,615

(22) PCT Filed: Jul. 28, 2008
(86) PCT No.: PCT/US2008/009094

§ 371 Date: Sep. 28, 2010

Related U.S. Application Data
(60) Provisional application No. 60/952,143, filed on Jul. 26, 2007.

(51) Int. Cl. C12Q 1/70; G01N 30/00; G01N 33/53; G01N 33/553; G06Q 50/00

(52) U.S. Cl. 435/5; 422/69; 435/7.2; 435/287.1; 436/526; 705/2

(57) ABSTRACT

This invention relates generally to a process for producing machine readable contextual diagnostic information and use of contextual diagnostic information for generating tangible and useful results. The process provides the highest level of integration of diagnostic information collected in a distributed or centralized system comprising diagnostic devices and a global computer network. More particularly, in certain embodiments, contextual diagnostic information are used in specific diagnostic related applications and business models including ecommerce.

### RELATED APPLICATION

This application claims the benefit of PCT/US2008/009094, having an international filing date of Jul. 28, 2008, which claims the benefit of U.S. Provisional Patent Application No. 60/952,143, filed Jul. 26, 2007, now expired. The entire teachings of the above applications are incorporated herein by reference.

### BACKGROUND OF THE INVENTION

Diagnostics, and more specifically, in vitro diagnostics, are an intrinsic and valuable element of quality health care. Their applications and contributions to quality of care are expansive across all aspects of patient care. Beyond the value to individual patients, diagnostics also contribute to the nation's hospitals, health systems and networks, and public health. Diagnostic tests serve a role in the measurements and tracking of standards and quality of care of health services by health insurers and healthcare systems, underlying their essential and multifunctional contribution to patient care. In addition, the protection of the public's health relies on diagnostics to detect infectious diseases such as SARS and West Nile virus and potential bio-terrorism threats such as botulism, anthrax, and small pox.

Diagnostic information is indispensable for decision-making by patients, clinicians, health care providers, healthcare purchasers, and public health officials. The decisions are made for patient specific and population wide health care treatments, measures, procedures, and services. Diagnostics provide key and sometimes critical information at multiple junctures along the health care continuum, from risk assessment and early diagnosis, to patient follow-up and disease management. The principal uses of diagnostics include diagnosis, primary risk assessment (i.e. predictive and early disease identification), prognosis, therapeutic selection, and disease or condition monitoring and management.

Diagnostics provide clinicians with information essential to making appropriate treatment and patient care decisions. In diagnosis, one or multiple tests are used, typically in combination with patient history and health practitioner experience, to identify a particular existing disease or condition. Some tests or test combinations may identify co-morbidities in addition to the primary diagnosis, providing information that can inform selection among alternative treatments or adjusting a treatment regimen.

Diagnostics can detect nascent disease or determine which patients are at increased risk for developing certain diseases (e.g., breast cancer, colorectal cancer). Determination of increased risk may allow patients and their health care providers to take measures to prevent or reduce the risk of developing a disease or condition, including increased medical monitoring, lifestyle changes, and preventive interventions.

Detection of emerging disease before symptoms appear or at early symptomatic stages allows significant opportunities for early prevention and treatment. Accurate and early detection and identification of diseases enable assessment of health status that can translate into reduced morbidity and mortality, improved quality of life, and reduced treatment costs. For example, early detection of colorectal cancer is associated with more successful treatment and increased survival rates. Diagnostics are evolving continually to enable more sensitive and specific detection of disease at earlier stages via measurement of biological chemicals, proteins, metabolites, and infectious organisms. Today, molecular diagnostics and other gene-based tests are emerging that enable the identification of susceptibility to disease long before symptoms occur. These diagnostics offer new opportunities for timely disease prevention and treatment.

The genetic profile or other biological predispositions of a patient may influence the individual's response to a drug. Emerging pharmacogenetics, pharmacogenomics, and molecular diagnostics use information about genetic variability to allow targeted treatment selection tailored to individual needs. Pharmacogenomic diagnostics are gene-based diagnostic tests used to determine the individual benefits or harms of taking certain medications. The knowledge of targeted treatments can allow health practitioners to avoid prescribing potentially harmful or ineffective treatments for patients, resulting in improved patient health outcomes and cost savings resulting from more effective health decision-making. Pharmacogenomics is contributing to an ideological shift within the medical community from a “one size fits all” drug treatment approach to that of “right amount of the right drug for the right patient.” Databases that compile and present such information are becoming available for scientists to study and clinicians to understand how genetic variations may relate to treatment outcomes. As the use of pharmacogenetics data becomes more integrated into clinical practice guidelines, electronic medical records, and decision support systems, clinicians will increasingly include pharmacogenomics in routine treatment decisions. The increasing use of pharmacogenomics holds great potential to yield better treatment selection and disease management strategies.

Diagnostic tests also may be used to assess the degree of disease progression or severity and the likelihood of recovery or risk of future adverse health outcomes. The prognostic information frequently is used to inform treatment decisions tailored to individual patient health status and needs. Prognostic assessment can include testing for certain co-morbidities (e.g., hypertension, cardiovascular disease, and diabetes). The presence of co-morbidity may inform necessary alterations in treatment options and therapeutic regimen.

Certain chronic diseases require continuous monitoring to avoid serious disease or treatment complications, maintain safe and effective levels of therapeutic drugs and screen for emerging resistance to medications or co-occurring infection (e.g., sepsis) or other diseases. Commonly used for these purposes, diagnostics are instrumental in helping clinicians and patients manage complex, currently incurable or later-stage diseases or conditions. Effective disease monitoring and management often is linked to reduced health care utilization, health care costs, and improved patient quality of life.

Point-of-care testing (POCT) or near-patient testing allows physicians to conduct rapid diagnostic tests while the patient waits, rather than sending samples to hospital or other centralized laboratories. Such rapid diagnostics provide health practitioners with information on patient health status and care options during the office and hospital visits. This immediate responsiveness reduces delays in effective health decision-making, allows rapid response to critical situations such as heart attacks, as well as routine and non-critical situations, and can reduce downstream health care costs.

Consumer expectations for diagnostics, such as rapid results, increased automation, simpler operation, and enhanced portability continues to drive the development of POCT devices. Next generation POCT diagnostics will incorporate evolving technologies such as nucleic acid amplification techniques, microarrays, and multiplexing. POCT diagnostics will continue to play a significant role in health decision-making, particularly in areas where rapid and accurate response is closely tied to health outcome such as the diagnosis of heart attack, the assessment of trauma patients, and the identification of certain infectious diseases including antibiotic resistant strains. POCT is particularly important where ready access to testing can improve patient compliance and continuity of care. Advances in POCT technology also have opened the door to home applications. Diagnostic tests can be completed in the home, or they can involve self-collection of blood, saliva, urine or other specimens that are shipped directly to the manufacturer or a reference laboratory for analysis. The Internet is becoming a convenient tool for sending, providing, and storing these test results for patients.

Public health, environmental, and bioterrorism-related diagnostics are used to detect infection or disease in individuals and for tracking population-level outbreaks. In public health, diagnostics have an array of applications, including population-level genetic screening of newborns, rapid identification of pathogens in disease outbreaks, identification of organisms that have developed antimicrobial resistance, and determining risk of future epidemics. As novel diagnostics continue to emerge in this area, public health threats can be characterized and contained more quickly and efficiently, affecting fewer individuals in improving public health management options. Use of diagnostics for these applications informs appropriate treatment and containment efforts to reduce the spread of infection. Diagnostics development in this area has focused on rapid and accurate results, as well as portable, easy-to-use instruments. Technological advances in these have great potential for cross-over into other segments of diagnostics and health care more broadly, increasing flexibility and responsiveness to changing health care needs.

Response to bioterrorism and/or an infectious disease outbreak represents a specialized area within public health that presents unique challenges and considerations. Here diagnostics contribute to two key factors; rapid detection of a causative pathogen or toxin and the initiation of proper containment and treatment measures. Newly developed rapid detection diagnostics may help decrease the time between introduction of a pathogen and detection, enabling faster and more effective threat response. Many of these emerging diagnostics also are being adapted for field use in emergency situations, ideally allowing containment efforts to begin before an infected person enters a health care facility.

In environmental/public health applications, diagnostic tests include monitoring biological and chemical levels in water and soil, surveillance of disease among marine and land animals and controlling growth of certain microorganisms potentially harmful in large numbers. Toxic biological or chemical agents in water or soil also may become incorporated into human food sources and have detrimental downstream effects on public health and national or global economies. Assessing the health of various marine and land animals also may be vital to protecting the agricultural supply chain and human health.

Monitoring livestock using emerging diagnostics offers opportunities for rapid identification of disease and timely response before unnecessary harm to livestock and subsequently to humans. Given the critical links between certain environmental chemicals and biological agents and human health, improved surveillance and control of these agents will translate into fewer incidents of disease in humans.

Emerging technologies, such as DNA microarrays, protein microarrays, and real-time PCR, are useful for associating expression of various biological products/biomarkers with health status or disease. As new biomarkers are validated, and as the significance of various combinations of biomarkers is better understood, these technologies are being adapted rapidly for a range of diagnostic applications. One expanding trend is multiplexing which involves conducting tests for more than one biomarker in the same test sample. This testing paradigm also is being developed in array formats, where multiple multiplex tests can be performed on the same platform or chip. Multiplexing has ushered in a new group of diagnostics which combine, for instance, the identification of infectious disease pathogens and (drug resistant) strain identification to allow clinicians to prescribe the most effective antimicrobial agent.

As diagnostics become increasingly integrated and capable of generating vast amounts of data, analytical advances and ease of interpretation will better facilitate adoption and diffusion of these technologies into routine clinical practice. For example, interpretation of a genetic or biomarker assay that includes several hundred tests may be too complex for use in general medical practice without software or information processing capabilities to assist with analysis and presentation of diagnostic results. As products that identify many hundreds or thousands of markers from a genome emerge, sophisticated analytical tools will be necessary to decipher the relationships between genetic makeup and predisposition to disease.

Advances in electronic medical records and decision support software, will assist clinicians in extracting meaning from increasingly complex diagnostic results. Computerized systems are currently assisting with processing certain laboratory tests, and similar systems will decrease diagnostic interpretation time, allowing for more rapid translation into appropriate prevention or treatment efforts. To the extent that information systems for health care providers can keep stride with advances in diagnostic throughput, these technologies hold the potential to dramatically augment patient care delivery.

The impact of diagnostics is undisputed as an essential role in the health and welfare of an individual, the general population, the food supply, and the environment. The significance of diagnostics is anticipated to increase as it evolves with other interventions and with health information technology. Web-based interfaces are anticipated to better link together the spectrum of patients, clinicians, health care providers, healthcare purchasers, and public health officials. The new diagnostic interfaces have the potential to redefine, on a global scale, the relationship among these participants through timely and relevant information available at the “finger tip.” The convergence of diagnostics and health information technology is driving the development of more rapid, accurate and high throughput diagnostic information. In some instances, future diagnostic devices will have a need to incorporate advanced information technology with greater integration capabilities, ease of use, and compatibility with other instruments or information resources (e.g., electronic medical records, health databases). Despite current advances, portable diagnostic devices, for example, point of care instruments or field use devices, still lack features and capabilities to further enhance the ability for patients, clinicians, health care providers, healthcare purchasers, and public health officials to capture, interpret, and use diagnostic information with greater speed, precision, and context. Conventional diagnostics provide minimal information such as a value concentration of a specific analyte to a patient or clinician but minimal contextual information as to the potential relevance of the result at a higher plane of observation. The majority of conventional diagnostics also fall short of providing rapid real-time assay results which is an essential element for time critical diagnostic decision-making.

The recent technology development using NMR detection with, for example, superparamagnetic nanosensors has enabled rapid and highly sensitive diagnostics for a wide variety of analytes and biomarkers. The capabilities of this diagnostic technology platform, for example, ultra-sensitive, homogeneous assays with rapid time-to-results, when combined with advances in computing, telecommunications, and satellite technologies will enhance the capacity and the potential of diagnostics, setting a stage for a paradigm shift in the generation and management of personal and epidemiological health information and services.

### SUMMARY OF THE INVENTION

In the broadest terms, the present invention provides a fee-based process for the generation of machine readable diagnostic information, making that diagnostic information available to a user and charging that user a fee for the diagnostic information. In some instances, the diagnostic information is contextual diagnostic information. There are several possible aspects to the invention each of which would involve a transaction step in some form. The transaction step is facilitated by a diagnostic technology platform, for example, a portable diagnostic device capable of performing and providing rapid and ultra-sensitive assay results for a wide variety of analytes and biomarkers combined with state-of-the-art computing and telecommunication technologies. A preferred diagnostic detection platform is described in greater detail in co-pending patent application Ser. No. 11/513,503, filed Aug. 31, 2006 (now U.S. Pat. No. 7,564,245, issued Jul. 21, 2009), No. 60/857,742, filed Nov. 8, 2006 (now Ser. No. 12/514,250); No. 60/904,685, filed Mar. 2, 2007, No. 60/919,236, filed Mar. 21, 2007, No. 60/915,797, filed May 3, 2007, and No. 60/912,298, filed Apr. 17, 2007, each incorporated herein by reference.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1 and 2 are schematic views of embodiments of the present invention method and systems.

FIG. 3-5 are schematic views of example deployments of various forms of contextual diagnostic information of the present invention.

FIG. 6 is a schematic view of a computer network environment in which embodiments of the present invention are deployed.

FIG. 7 is a block diagram of a computer node in FIG. 6.

### DETAILED DESCRIPTION OF THE INVENTION

A description of example embodiments of the invention follows.

The term “diagnostic device” as used herein, refers to a device or substance used for diagnosis, primary risk assessment (predictive and early disease identification), prognosis, therapeutic selection, disease or condition monitoring and management, population genetics screening and monitoring, pharmacogenomic diagnostics, epidemiological studies and monitoring, clinical trials monitoring, and syndromic surveillance including clinical analyzers, portable battery operated meters, self-performing assay devices, point of care analyzers, point of care meters, point of present analyzers, point of present meters, etc.,

The term “analytes” as used herein, refers to an atom, an ion, a molecule, a compound, a catalyst, an enzyme, an electroactive mediator, an electron-pair donor, an electron-pair acceptor, a lanthanide, an amino acid, a nucleic acid, an oligonucleotide, a polymer, an aptamer, a therapeutic agent, a biological molecule, a metabolite of a therapeutic agent, a peptide, a polypeptide, a protein, a carbohydrate, a polysaccharide, RNA, DNA, RNAi, an antibody, an organism, a virus, bacteria, a prion, a carbohydrate, a polysaccharide, a lipid, a gas (e.g., oxygen, carbon dioxide), a constituent of a clinical chemistry panel including electrolytes (e.g., sodium, potassium, chloride, bicarbonate, BUN, creatinine, glucose, magnesium, phosphate, calcium, ammonia, lactate), a lipoprotein, cholesterol, a fatty acid, a glycoprotein, a proteoglycan, and/or a lipopolysaccharide, a virus components (i.e. capsids), a cell, components of cells (i.e. vesicles, apoptotic bodies, organelles, cell debris/dead cells), and other particles (i.e. circulating clots, cholesterol particles, plaques, forms of amyloid, and micelles), a prokaryotic cell such as bacteria or an eukaryotic cell such as mammalian cell including cells of a human organ, a surface antigen, a G-protein receptor, an infectious disease agents, E. coli, botulism, Ebola virus, bubonic plaque, Methicillin resistant Staphylococcus aureus and their variants, Vancomycin resistant Staphylococcus aureus and their variants, any antimicrobial resistant bacterial and viral strains, nerve agents, blood agents, blister agents, pulmonary agents, incapacitating agents (e.g. lachrymatory agents), cholera, tularemia, brucellosis, Q fever, typhoid, tuberculosis, influenza A, influenza B, hepatitis A-E, HW and variants, encephalitis, smallpox, ricin, SEB, botulism toxin, saxitoxin, mycotoxin, and/or other toxins, SARS, sexually transmitted disease agents (i.e. Chlamydia Gonorrhea, Herpes Simplex, Syphilis, Trichomonas), causative agents of sepsis, environmental pollutant, and any atom, ion, or molecule that antibody can be produced using known immunological methods and combinations thereof.

The term “biomarkers” as used herein, refers to anatomic, physiologic, biochemical, or molecular parameters associated with the presence and severity of specific disease states and includes cancer biomarkers (i.e. PSA, etc.), cardiovascular disease biomarkers (i.e. troponin, CKMB, myoglobin, etc.), therapeutic drug monitoring biomarkers, etc.

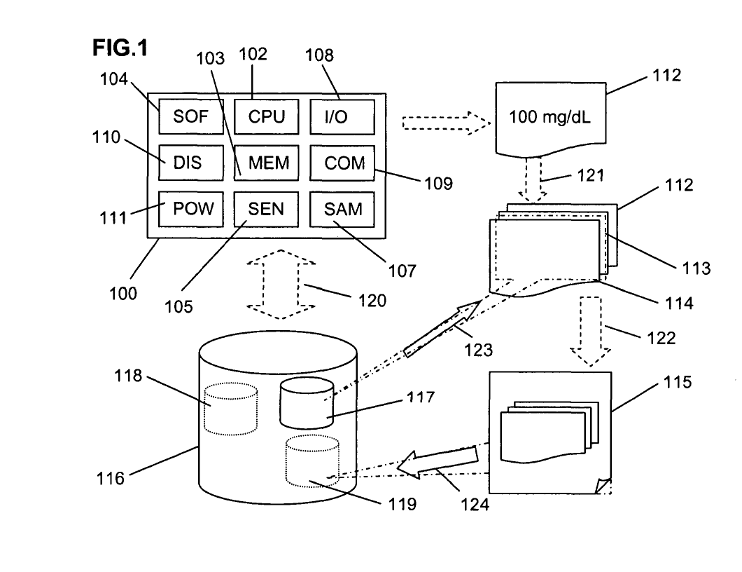

One aspect of the invention is providing real time diagnostic information to a health care provider in a point of care setting such as a doctor in a doctor's examining room or an emergency medical technician (EMT) in an ambulance and charging a fee for the assay. In that sense, the present invention provides the commoditizing of real-time (near real-time) assays and diagnostic information. The invention deployment of fee for real-time/near real-time diagnostic information and analytical assay is specific and distinct from fee for services by physician and EMT and the accounting thereof. In a preferred embodiment illustrated in FIG. 1, the diagnostic device 100 is a portable diagnostic instrument capable of generating an analytical result 112. The diagnostic device 100 comprises a microprocessor unit 102 for executing machine readable software instruction 104 stored in a memory device 103. The diagnostic device 100 contains a test sample processing module 107 that operates in conjunction with a sensing module 105. The sensing module 105 can include one or more test sample sensors and augmentation parameter sensors, for example global positioning system (GPS) sensors. A communication module 109 can be included within the device 100 or operate in combination with the device 100 through an I/O module 108. In this particular embodiment, the communication module 109 is contained within diagnostic device 100. The I/O module 108 can receive user input using a variety of input devices including keypads or touch screen. The diagnostic device 100 also has a display 110 for a user-interface, a communication module 109, and a power source 111. The communication module 109 contains electronic components as to enable wired or wireless communication to a device external to the diagnostic device 100. For example, the communication module 109 can receive commands from an external computing server 116 or send analytical result 112 to other external devices. The said components are configured as to enable the diagnostic device 100 to perform test sample acquisition, processing, detection, analysis, and generation of an analytical result 112. As an example, the analytical test result 112 can be a concentration value of the biomarker troponin obtained by processing a blood sample using the test sample processing (sample preparation) module 107 and detecting the presence of troponin using a biosensor in the sensing module 105. The test result can be generated in the presence of a patient during transport to a hospital in an ambulance.

Another aspect of the invention includes providing the diagnostic information 112 to a central database 117 from which it can be accessed by a health care provider that is not within the immediate vicinity of the patient such as a primary care physician who has a patient being transported by ambulance to a local or remote medical facility and charging a fee for the assay and/or the health care provider's access to the database 117. In a preferred embodiment, the invention includes providing (outputting) the diagnostic information 112 to a central server 116 which then electronically notifies a particular healthcare provider that his or her patient has had one or more diagnostic tests performed on a sample of the patient's and where this was done such as a physician being notified that his or her patient has had tests related to a possible coronary event done in an ambulance that is en route to a medical facility and charging a fee for the assay and/or the notification to the health care provider.

In another preferred embodiment, an analytical test result 112 generated by the diagnostic device 100 is combined with one or more augmentation parameters 113,114 resulting in a contextual diagnostic information unit 115. The augmentation parameter 113,114 can include for example a geographic location of the diagnostic device 100 or additional information providing a context for the analytical test result 112.

To further illustrate, an augmentation parameter 113,114 is one or more parameters that enhance the decision-making process for a spectrum of participants in a medical related interaction. In the example of a patient with symptoms suggestive of a myocardio infarction (AMI) event, a blood test of the patient is performed using a diagnostic device 100. While in transit to a hospital, a diagnostic device 100 conducts an assay and obtains an analytical result 112 with a concentration value of troponin indicative of an AMI. Upon obtaining an analytical result 112, the diagnostic device 100 appends (at 121) one or more augmentation parameters 113,114 such as the position/location or velocity of the diagnostic device 100 sensed by the device sensing module 105, within the ambulance transporting a patient to an emergency room.

The diagnostic device 100 can also communicate with computing server 116 (such as at 120), either manually or automatically, to obtain additional augmentation parameters 113,114 such as frame-of-reference data, contained in one or more databases 117,118, append the additional information to the test result 112 (steps 121,123) and transmit the combined information 115 back to a computing server 116 (steps 122,124), and store the result in another database 119. The analytical test result 112 and one or more augmentation parameters 113,114 are combined (through steps 121,122,123) into a contextual diagnostic information unit 115, which is transmitted (step 124) to server 116, and then subsequently stored in a database 119. The contextual diagnostic information unit 115 becomes useful upon presentation to health care providers. For example, the information available from the computing server 116 can allow an emergency team of a hospital to prepare the catheterization laboratory for reception of the patient arriving in the ambulance by tracking the ambulance.

In addition to the emergency team, a surgeon remote from the hospital can be alerted by phone, text message, paging and the like that a patient will be arriving and being prepped for surgery. The techno-savvy surgeon may use a portable device capable of web-browsing and searching to access a computing server 116 to track the position and velocity of the arriving patient in real-time. The surgeon can coordinate his arrival with the arrival of the patient. The enhanced decision-making process for the emergency team can be the preparation or assembly of the necessary experts for an arriving patient or diverting the ambulance to an appropriate facility capable of treating a patient, thus implementing and providing the most efficient health service available to the patient. The surgeon can best track the arrival of a patient having the contextual diagnostic information 115 and make a decision on the best utility of his resources.

The contextual diagnostic information 115 can be selective with one or more augmentation parameters 113,114 that are most useful or best appropriate to a particular scenario or application or most relevant to the context of the generation of an analytical test result 112. In addition, the preferred diagnostic device is capable of generating rapid, point of care, ultra-sensitive analytical results as to enable decision-making with speed and precision for time-critical diagnostic related events including medical diagnostics and the potential spread of an infectious disease.

An additional aspect of the invention includes integrating the diagnostic information 112 with additional data and making that data available to parties interested in the resulting contextual diagnostic information 115 such as CDC officials who want to know where cases of a particular disease have been identified and in what time frames and charging a fee for the assay, the analysis and/or the user's access to the diagnostic information 115.

A further aspect of the invention includes integrating the diagnostic information 1 12 with additional data 113,114 and performing an analysis to determine various parameters of interest and providing the resulting contextual diagnostic information to relevant parties such as possible future trends for a chemical or biological agent that has been released into the air within a metropolitan area, or a city, or a park, or within an airport, or the environment.

Contextual diagnostic information 115 includes an analytical result 112 combined with additional information 113,114 to enable, for example, optimal decision-making. Such information can be attributes or characteristics pre-loaded in a diagnostic device 100, or attributes measured by sensors 105 of the diagnostic device, or attributes generated from a source external to the device 100. The additional information is represented in the present invention system as augmentation parameters 113,114. The augmentation parameters 113,114 can include, for example, position, velocity, or acceleration, or geospatial position of the diagnostic device 100 that is generating the analytical result 112. Such information can include, for example, information about the external environment of where the analytical result was generated such as wind speed and direction. Such information can also include financial information relating to an analytical result 112, for example the monetary value of the analytical result.

The diagnostic devices 100 can be distributed (of a distributed processing network form) or ambulatory diagnostic devices capable of wired or wireless communication with a computing server 116. The computing server 116 can be accessible through an intranet, or global computer network, e.g., Internet, or the World Wide Web. The computing server 116 includes hardware and software for transmitting, receiving, processing, and transforming machine readable diagnostic information generated from one or more diagnostic devices 100 or from other sources of diagnostic information. The machine readable diagnostic information contained within a database 117,119 of the computing server 116 is web-accessible using a search engine or other form of information retrieval software.

In some instances, the computing server 116 enables diagnostic information 112,115 and information gathered from other databases accessible via an intranet, Internet (generally, global computer network), or World Wide Web, to be analyzed and interpreted using web-accessible information representation formats, e.g. web browser. By combining diagnostic information 112 with additional information 113,114 that adds context or perspective to the diagnostic information one can obtain a selective level of detail for diagnostic-related health events including local, global, temporal-spatial, financial, social, implications of diagnostic-related health events in either a textual or graphical representation. The representations can be used for at least one of the following tasks including diagnosis, primary risk assessment (predictive and early disease identification), prognosis, therapeutic selection, disease or condition monitoring and management, population genetics screening and monitoring, pharmacogenomic diagnostics, epidemiological studies and monitoring, clinical trials monitoring, alert function of the ER, and syndromic surveillance relating to public health, the environment, and bio-terrorism. The term “syndromic surveillance” applies to surveillance using health-related data that precede diagnosis and signal a sufficient probability of a case or an outbreak to warrant further public health response. Though historically syndromic surveillance has been utilized to target investigation of potential cases, its utility for detecting outbreaks associated with bioterrorism is increasingly being explored by public health officials.

In one embodiment, machine readable contextual diagnostic information 115 is generated from a diagnostic device capable of providing rapid analytical results using ultra-sensitive detection technology and one or more external sources of additional information. The diagnostic device 100 can be a portable NMR relaxometer capable of providing rapid and ultra-sensitive detection of analytes. It can be used to determine discrete or continuous in vitro or in vivo analytical results from a sample. The in vivo application of the diagnostic device can be either invasive or non-invasive. The sample can include at least one analyte solution, aerosol, environmental source (i.e. water, air, etc.), biological fluid, analyte of a biological organ, biomarker of a biological organ, analyte of an animal, biomarker of an animal, or analyte of a human, or biomarker of a human, or a causative agent of disease. Contextual diagnostic information 115 includes at least one analytical result 112 such as a value or changes in a value of an amount or concentration of a specific molecule or analyte of detection and one or more additional items of information 113,114 of relevance to the analytical result. The additional information 113,114 can include one or more of the following: device unique identifier, the location, position, velocity, or acceleration of the device 100, a patient biometric, a patient demographic, a specimen genetic profile, a therapeutic agent, an illicit drug, adverse-drug reaction information, drug interaction information, a critical value range for one or more analytes, a list of chief complaints from which the user of the device can choose, a list of potential types of observations, healthcare provider annotations, temporal-spatial information, a listing of event frequencies or statistics for the patient or area or for a general population or control area, result trends, geospatial and geostatistical information, temperature, wind speed, wind direction, barometric pressure, geospatial coordinates, the internet protocol address(es) assigned to the diagnostic device, the monetary value assigned to the diagnostic information 112, charge centers or other information related to who should be charged for the diagnostic information 112 or searchable events relevant to the specific analyte of detection extracted from web-pages, text messages, emails, and blogs that are either directly related to the patient or to a larger group or area.

The additional information 113,114 can be a parameter pre-programmed in the diagnostic device 100, or manually input into it by the user. The information can be collected from sensors external and communicable to the device or from one or more database accessible using wired or wireless communication through an intranet, Internet, or the World Wide Web. The generation of contextual diagnostic information 115 can be performed by a user by manual input or by queries through a software user-interface. Alternatively, a computing server 116 can enable a user to control the operation of a diagnostic device 100 at a location that is remote from the user. In some embodiments, the user can command the diagnostic device 100 to obtain an analytical result 112, send and receive queries, or append additional information 113,114 of choice to an analytical result for subsequent transmission and reception of the contextual diagnostic information 115 over a wire, wireless, telecommunication system, an intranet, the Internet, or the World Wide Web to a database 119 of the computing server 116. The machine readable contextual diagnostic information 115 can be stored in a format that is indexable and searchable.

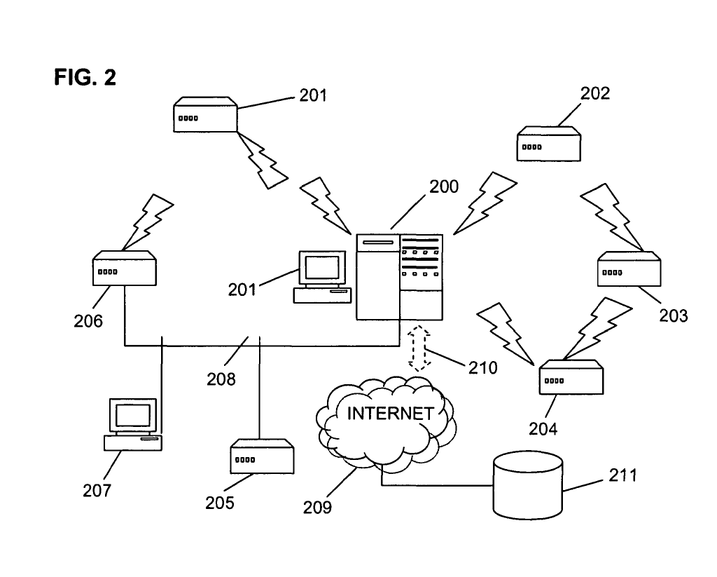

In a preferred embodiment as shown in FIG. 2, the diagnostic devices 100 (shown at 201, 202, 203, 204, 205, 206) are distributed freely in a communication network and its relation to the global computer network (e.g. the Internet). Central to the diagnostic device network is a computing server 200 accessible to a local user with a computer terminal 201. The computing server 200 is also accessible to a user at a remote computer terminal 207 connected the network via wired communication system 208. The computing server 200 is in communication with one or more stationary diagnostic devices 206,205 and one or more ambulatory distributed diagnostic devices 201,202,203,204. The distributed diagnostic devices may be deployed at various global locations such as a port of entry (i.e. airport security entrances) or within a local environment such as a hospital complex (i.e. ICU). The ambulatory diagnostic devices 201, 202, 203, 204 are capable of communicating with computing server 200 through a wireless telecommunication system. In addition, ambulatory diagnostic devices can communicate with each other through a wired or wireless telecommunication system. The computing server 200 enables users to access at least one index-able and searchable database 211. In addition, the diagnostic devices 201, 202, 203, 204, 205, 206 can also access one or more databases 211 through the computing server 200. The one or more index-able and searchable database can contain frame-of-reference information for the network of diagnostic devices. The frame-of-reference information can be a geographical information system (GIS). The one or more index-able and searchable database 211 can be any database connected to an intranet, or global computer network (e.g. the Internet, or the World Wide Web). The computing server 200 enables one or more users and one or more diagnostic devices 201, 202, 203, 204, 205, 206 to send, receive, and process contextual diagnostic information generated by the diagnostic device network. The computing server 200 contains software for processing contextual diagnostic information including extraction, translation, and loading to establish a web-based contextual diagnostic information data mart. The data mart is accessible to a spectrum of users using for example a web-browser or similar application software.

In a detailed example directed to the generation of a geo-position augmentation parameter, a diagnostic device 201 receives the position signals from at least three satellites of a telecommunication network, calculates a self position, appends the calculated position to the analytical result, and sends the mapped position to a computing server 200. The diagnostic devices 201 perform wireless communication through the wireless communication network and connect with the Internet 209 (global computer network) to perform Internet services. For example, using one or more databases 211, the computing server 200 maps a universal resource locator (URL) registered to the Internet to each of the icons and names for the peripheral facilities and the mapped position of a diagnostic device 201 on a map and stores the information on another database or database 211. In an alternative scenario for generation of positional augmentation parameter, the computer server 200 and one or more diagnostic devices 201 can be part of mobile communication network. The mobile communication network can include a plurality of base stations and wireless connection with the diagnostic devices. The computer server 200 can be located at a base station connected to a mobile switching center for connecting with other communication networks or forming a connection point for communication with other mobile switching centers, and a gateway connected with the Internet 209. The positional augmentation parameter for the diagnostic device 201 is generated using triangulation methods in conjunction with cellular towers and base stations.

The fee to be charged for providing either diagnostic information or contextual diagnostic information is done as part of a transaction as a fee for services or fee for information. As used herein, “transaction” is understood to mean an identifiable operation carried out by or through a machine, or individual, or an organization that transforms or converts an asset. Such a transaction can take place in one or more device-user, “brick-and-mortar”, or ecommerce business models including advertising, and subscription. The fee may be charged directly to the user (i.e., a patient), to an entity that wants access to the user (i.e., an advertiser) or to other types of entities such as insurance companies, employers, local, state or federal government etc.

As an illustration, a process of the present invention uses machine readable contextual diagnostic information to produce tangible and useful results in one or more business models. In a world comprising a number of market participants, contextual diagnostic information produced from one or more processes of the present invention, mediates a transaction, through at least one asset specificity, defining a product or service to be exchanged by two or more participants. A transaction can also take place between a single participant and another device such as the diagnostic device of the present invention. The asset specificity enables contextual diagnostic information to be monitized, for example, providing useful information through an information generating device that minimizes transaction costs for at least one participant. In exchange for minimizing the transaction costs for at least one participant, one or more fees are produced and charged for mediating a transaction. The transaction can take place in one or more device-user, “brick-and-mortar”, or ecommerce business models including advertising, affiliate, broker, community, infomediary, manufacturer, merchant, utility, and subscription. The asset specificity is preferably conferred from one or more contextual diagnostic information, which makes a necessary contribution to the provision of a medical diagnostic product or service and has a significantly lower value in alternative uses. The asset specificity can be one or more analytical results (i.e. a concentration of an analyte) generated within 15 minutes, preferably within 5 minutes, more preferably within 1 minute, most preferably in real-time and with high-sensitivity or ultra-sensitivity (i.e., single molecule, cell, viral particles, etc.) and/or with augmentation parameters of relevance to a stationary point of care setting (i.e. home, physician's office, hospital, bedside, ER, ICU, OR, retail store, etc.) or field location (i.e. airport, harbor, parks, forests, farm, desert, plains, etc.) or within an ambulatory/motional vehicle (i.e. car, ambulance, airplane, a helicopter, un-manned vehicle, robot, trains, missile, rocket, space craft, etc.). The diagnostic products or services enable one or more computer-assisted tasks including diagnosis, primary risk assessment (i.e. predictive and early disease identification), prognosis, therapeutic selection, disease or condition monitoring and management, population genetics screening and monitoring, pharmacogenomic diagnostics, epidemiological studies and monitoring, clinical trials monitoring, alerting, and syndromic surveillance relating to public health, the environment, and bio-terrorism.

In another embodiment of the invention, one or more diagnostic devices 100, 201, 206 form a distributed network of machine readable contextual diagnostic information portals. The one or more diagnostic devices 100, 201, 206 perform one or more analytical assays. The one or more diagnostic devices are connected to a centralized computing web server 116, 200 comprising at least one indexable and searchable database 117, 118, 211 via a wired or wireless telecommunication system, including an intranet, the Internet and the World Wide Web or any other global computer network. The one or more indexable and searchable database can contain frame-of-reference information for the contextual diagnostic information. The computer-assisted web server can receive contextual diagnostic information and process the information including extracting, translating, and loading the contextual diagnostic information to a database to establish a contextual diagnostic information data mart with web-based reporting. The web server can be connected to an intranet, the Internet, or the World Wide Web or other global computer network to allow controlled access by a spectrum of users.

In a preferred embodiment, the present invention has a computer- implemented method for using a search engine to search web pages and any electronic database in response to an Internet-based search query. The searchable database 117, 118, 211 includes electronic versions of printed or electronic media stored in a memory bank and coupled to a computer node of the Internet 209 (global computer network generally). The searchable database 117, 118, 211 can also be stored in multiple memory banks that are located at various nodes along the data network and coordinated logically to operate as a unified memory arrangement. The computing server 116, 200 enables users to search and find relevant items using both a web-type search effort for locating documents that one would expect to find via an Internet-based search. These types of items are stored as respective data sets and are stored in a searchable electronic database. A user, or other input source, prompts the computing server 116, 200 with a search query. In response, the computing server 116, 200 then electronically searches both the web-accessible documents and contextual diagnostic information data sets for relevant items. Returned as search results are characterizations of any relevant web-accessible documents and of any respective data sets and an electronic path for accessing further information. The electronic path permits access to more characterizations of the relevant items such as to information for permitting a subscription-like access.

The computing server 116, 200 of the present invention has a memory bank arrangement that is adapted to store and maintain the searchable database as data sets. The database can include application software necessary to organize and relate the stored information, making the information retrievable. Each data set can include text, graphics, or both from one of the printed or electronic media items. In one implementation the database is a relational database and in another implementation, the database is a library of hyperlinked documents hosted on at least one web page server coupled to the data network.

The Web pages, or hyperlinked documents, of the present invention are hosted by at least one web page server. A user accesses and navigates the data network through a computer terminal coupled to the data network and an application program, typically referred to as a browser. Another programmable computer node includes a search engine application, the search engine being accessible by the user through the user's browser application and the data network. The user enters a search query, for example a keyword search, and the search engine is adapted to search portions of the data network responsive to the user's search request to identify data network (logical) destinations relevant to the user's search query. The search engine then returns for display results of the search including at least one characterization of a relevant web page destination, and at least one characterization of a destination data set representing a relevant printed or electronic media item. Each characterization includes an accompanying electronic path recognizable by the user's browser to navigate to a data network destination related to the respective search result for retrieval of additional information.

In a specific search example, the computing server 116, 200 presents a computerized nationwide photograph or satellite imagery and allows a user to “zoom in” on an area or even on a specific location on the map at a computer terminal 207. The aerial map may incorporate an overlay of contextual diagnostic data that allows the aerial map to provide special indications of diagnostic related events. In addition to diagnostic data overlay, many different types of data overlays may be applied to the aerial map information for example the local weather. In general, the overlay information may include any type of location-based data. The aerial map may also integrate various type of geographic vector and point data (e.g., shown as drawn-in information in the aerial map) so that streets, boundaries, and other information that are not evident from the aerial picture alone may be identified.

In some embodiments, the web computing server may employ a viewer that allows a user to zoom in on a map via a web browser without needing to download a specific client application. Any range of zooming may be implemented. For example, at the highest level, the map may cover an entire country or continent, or even the whole world. In some cases, the user may be able to set filters to include/exclude selective information.

In other embodiments, the computing server 116, 200 may handle many different data sources, although in its simplest form, it may handle only an underlying map layer made up of satellite imagery, aerial photo imagery, and/or the like. These imagery data sources may sometimes be referred to as “Digital Orthorectified Quadrangles” (DOQs). Digital orthography is the process by which images are adjusted to account for elevation changes so that aspects of the image can line up appropriately. For example, the United States Geological Survey (USGS) has been making high-resolution imagery of this type available on a city-by-city basis. Likewise, many private companies provide such imagery.

In yet another embodiment, aerial imagery is imported into an intermediary software tool. For example, one or more tools may break down large image files into many smaller files (e.g., “map tiles”) and generate an index file to help locate the many smaller map tiles. The map tiles may go through some additional postprocessing prior to use, such as coloring the water or re-coloring the map, or adding additional layers to the tiles. In some embodiments, map tiles need not be image files. Rather they can be any information/data that facilitates the electronic display of one or more maps. A single map displayed on a screen may be comprised of one or many map tiles.

In yet another alternative embodiment, the one or more software tools may generate such map tiles at many resolutions to enable effective zooming in/out. For example, each time a user zooms in on a map, a new set of map tiles (e.g., a three-by-three square of nine map tiles configured at a higher resolution and covering less geography) may replace an earlier set of map tiles (e.g., a three-by-three square of nine map tiles configured at a lower resolution and covering a greater geographical area).

Any number of overlays of the present invention may be added on top of the map layer, with each overlay including additional data of interest. In some embodiments, the overlays may comprise information based on vectors, points, or both vectors and points. For example, U.S. city names may be aggregated into a overlay that defines cities using points, which are then drawn as groups of pixels (or icons) on top of the aerial imagery to identify cities and associated information (e.g., airport, shipping dock, etc.). In another example, the points of interest information or select amenities information may provide similar overlays using points (e.g., identifying restaurants, airports, parks, shopping centers, zoo, etc.). In some cases, the user using filters or searching techniques may access these points of interest.

Likewise, some overlays may be based on vector information and may provide displays of lines (as in the case of roads) and polygons (parcel outlines, park boundaries, state boundaries, etc.) on top of a map layer. For example, neighborhoods information, boundaries information, roads information, bodies of water information, parks information, schools information, etc., may all be defined and illustrated using vectors. Additional vector layers may be added when available. For example, a county parcel map may be distributed by some county agencies and, when accurate, may be an appropriate overlay on aerial imagery. Like the points described above, these vector-based overlays may also be accessed by searching techniques (e.g., using keywords) or by filtering techniques, etc.

While specific types of layering/overlay schemes based on specific types of information are described above, almost any type of data that has any diagnostic relevance can also be tied to the aerial map, for example, cluster analysis and resulting data performed by another software module of the computing server. In some embodiments, the information used for overlays may be associated with more detailed information that may be presented when a user clicks on (selects) an object identified on a map. Likewise, it may be possible for the user to click (command) to add or remove different layers of data. Each layer may be represented by a different color or other attributes.

With respect to implementation of such overlays, in some embodiments, point and vector overlays can be delivered as database tables or flat files (e.g., ESRI shapefiles). Shapefile is the most common flat file format supported by nearly all Geographic Information System (GIS) data suppliers. In some embodiments, shapefiles are run through a software tool and imported into a database 117, 118, 211. To help with the matching up of map images to overlay information, the overlay information, which may be implemented using points and/or vectors, may be structured using a geographical coordinate system similar to the geographical coordinate system used in GIS maps. In this way, mathematical projections can be used to match overlays with maps, so that the overlay information lines up with the map information as accurately as possible.

Alternatively, in addition to the data scheme described above, the aerial image maps can be integrated with data from other data sources, such as third party data sources. Accordingly, there are few limits on what types of information can be shown on such maps. Some examples include source specific information about points of interest, weather information, news information, and so forth.

In yet another embodiment, the contextual diagnostic information 115 can be stored in one or more database 119, 211 of a computing server 116, 200. The computing server 116, 200 can be a centralized computing server, a distributed server, or another diagnostic device containing hardware and software sufficient to perform computing server functions. The contextual diagnostic information 115 within the database 119, 211 can be further augmented with other sources of information, including frame-of-reference data, or combined with other diagnostic modalities using software contained within the computing server 116, 200. A variety of software, for example, a web-browser, can be used with the computing server 116, 200 to allow remote users to access the contextual diagnostic information 115 for data transformation and analysis that enables local, global, temporal-spatial visualization and real-time decision-making. The transformation of contextual diagnostic information 115, can be selective and in certain cases confidential information may be modified according to specific or regulatory standards as to comply with the standard such as maintaining anonymity. The contextual diagnostic information, including other sources of information, can be used to perform at least one of the following computer-assisted tasks including diagnosis, primary risk assessment (predictive and early disease identification), prognosis, therapeutic selection, disease or condition monitoring and management, population-level genetics screening and monitoring, pharmacogenomic diagnostics, epidemiological studies and monitoring, clinical trials monitoring, alerting, and syndromic surveillance relating to public health, the environment, and bio-terrorism.

In another embodiment, the process includes accessing machine readable contextual diagnostic information 115 from at least one or more diagnostic devices 100, 201, 206 that can be controlled remotely. The accessed contextual diagnostic information 115 is analyzed to identify one or more features of interest available from the diagnostic device via a computer-assisted data analysis algorithm such as the frequency of a specific analytical result. The computer-assisted data analysis algorithm can be time series analysis, geostatistical analysis, trends analysis, artificial intelligence analysis, wavelet analysis, neural network, Kalman filtering, univariate statistical process control, multivariate statistical process control or combinations thereof. The features of interest can be stored in an indexable and searchable format. The stored features of interest can be combined, compared, merge, transformed, and evaluated with any other source of information to enable effective decision-making.

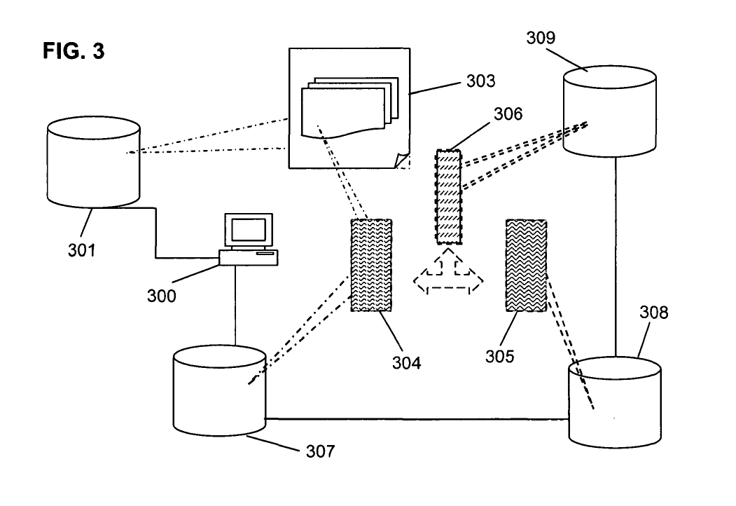

As illustrated in FIG. 3, various resources may be implemented within the process of the present invention to use contextual diagnostic information. One resource is a computing terminal 300 that has access to a computing server 116, 200. Using a computing server 116, 200 a user at a terminal 300 can download a variety of data stored in one or more databases 301, 307, 308, 309. One database 301 may contain contextual diagnostic information 303 (like 115). Another database 308 may contain one or more signature data set predetermined profiles of known medical conditions or an integrated knowledge base. The integrated knowledge base may include or be genetic information such as the human genome. The user at a terminal 300 can perform a wide variety of data analysis using for example contextual diagnostic information 303. The contextual diagnostic information 303 may be filtered or transform into another data form 304 and stored into a different database 307. For example, the contextual diagnostic information 303 may contain a patient's identification formatted in HL7. Using software contained within a computing server 116, 200 the contextual diagnostic information 303 is processed to strip privacy data from contextual diagnostic information 303 resulting in data form 304 which may be then stored on a database 307. The data form 304 may also be used to compare with a data set 305 stored on another database 308. The data form 304 may then be processed and correlated with data set 305 of patient-specific, or population-specific, or condition-specific resulting in another data set 306 which is then stored in data base 309. The one or more data bases 301, 307, 308, 309 and stored data sets can be used, as an example, for spatio-temporal pattern detection of a potential spread of communicable disease. Software applications such as S+SPATIAL or WinBUGS can be used for data processing and generation of statistic results. Proprietary software programs can be written incorporating various statistical methods for the analysis and generation of useful results. The statistic methods include CuSums, English Model, SPOTv2, time series analysis, Hidden Markov, scan statistics, and Kalman Filter, among others. The results are stored and accessible using a web-browser.

In yet another embodiment, a process is provided in which the machine readable contextual diagnostic information is analyzed with a predetermined profile for at least one known medical condition. The contextual diagnostic information is accessed and compared with a predetermined profile for the known medical condition using a computer-assisted data analysis algorithm to determine the likelihood that a subject has the medical condition. If necessary, subsequent data analysis can be performed based upon the results of the comparison.

In still another embodiment, a process is provided wherein in the machine readable contextual diagnostic information is accessed as is contextual diagnostic information from an integrated knowledge base including contextual diagnostic information derived from one or more diagnostic device that can be controlled remotely. The contextual diagnostic information and the accessed contextual diagnostic information are re-analyzed via a computer-assisted data analysis algorithm to identify at least one feature of interest of the contextual diagnostic information. The acquisition of an analytical result, processing of an analytical result or processing of data derived is modified based upon the comparison. The modified data is used to perform at least one of the following computer-assisted tasks including diagnosis, primary risk assessment (predictive and early disease identification), prognosis, therapeutic selection, disease or condition monitoring and management, population-level genetics screening and monitoring, pharmacogenomic diagnostics, epidemiological studies and monitoring, clinical trials monitoring, alerting, and syndromic surveillance relating to public health, the environment, and bio-terrorism.

The present invention provides a process for handling of machine readable contextual diagnostic information designed to enhance global diagnostic knowledge. The process can draw upon the full range of available medical data. Such medical data can be part of an integrated knowledge base, accessible through a telecommunication system including an intranet, the Internet, and the World Wide Web or any other global computer network. The integrated knowledge base, itself, can be analytically subdivided into certain data resources and/or other diagnostic devices. The data resources include databases which are patient-specific, population-specific, condition-specific, or that group any number of factors, including physical factors, genetic factors, financial and economic factors, and so forth. Based upon such data, routines executed by one or a network of computer systems defining a general processing system, can identify, diagnose, and alert users to potential medical events. Moreover, the processing system can suggest or prescribe additional data acquisition steps from the diagnostic devices, including different types of contextual diagnostic parameters during a single time period or over extended periods of time.

According to another embodiment, the present invention is directed to a machine-implemented method that searches World Wide Web-accessible health related and non-health information including media information, e-commerce transactions, Internet traffic and data packets. This information can be stored in a searchable electronic database. In response to a search query, the machine electronically searches the web-accessible documents that are relevant to the search query and searches the data sets in the electronic database for data sets that are relevant to the search query, thereby identifying web-accessible documents and relevant data sets corresponding to relevant health related and non-health information. The health related and non-health information are processed using a computer-assisted method to link health related and non-health information with searchable machine readable contextual diagnostic information according to a defined algorithm for correlating search query results from web-accessible documents with contextual diagnostic information. The search query results are returned in one or more web documents with temporal-spatial text and graphical information to allow varying levels of observation of local and/or global events in conjunction with contextual diagnostic information. The observation enables a user to perform at least one of the following computer-assisted tasks including diagnosis, primary risk assessment (predictive and early disease identification), prognosis, therapeutic selection, disease or condition monitoring and management, population-level genetics screening and monitoring, pharmacogenomic diagnostics, epidemiological studies and monitoring, clinical trials monitoring, alerting, and syndromic surveillance relating to public health, the environment, and bio-terrorism.

Consistent with the above computer-implemented methods, more specific embodiments are directed to search results of machine readable contextual diagnostic information and to producing with the search links of contextual diagnostic information to health related and non-health information that would permit subscription-like access. Subscription access also includes search results of further information about the relevant search query beyond the contextual diagnostic information. The search results can also include analyses and presentation of the contextual diagnostic information. The search results can also be analyzed to obtain e-commerce information including user search key-words, web page visits, the frequency of visits, user demographics, user biometric information, user preferences, surveys, blogs, chats, messaging, and emails. The search results may be employed for a number of purposes, first and foremost for the diagnosis and treatment of medical events, epidemiological trends, including syndromic surveillance of public health, environmental monitoring, and bio-terrorism. Thus, patient care, treatments, or crisis response can be improved by more rapid and informed identification of disease states, medical conditions, predispositions for future conditions and events. In addition, the system allows for more rapid, informed, targeted and efficient data acquisition, based upon such factors as the medical events or conditions which are apt to be of greatest priority or importance.

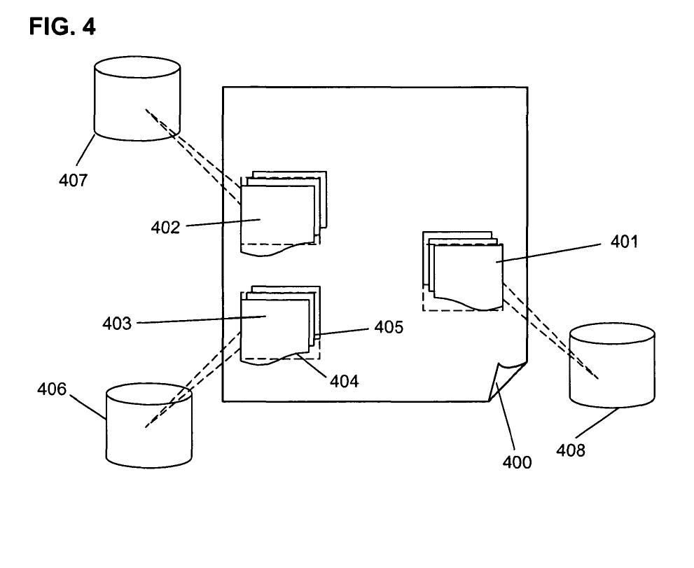

In a conceptual illustration in FIG. 4 of the computer-method for data acquisition of web-based information, one or more web page 400 stored in a database connected to the Internet comprises one or more information units 401, 402, 403. The information units can be data stored in on or more databases 406, 407, 408. The information unit can be text or graphical format. An information unit 403 can be comprised of additional information sub-units 404, 405. The information unit may be a contextual diagnostic information unit 115. One or more web pages can be, for example, Google News, ProMED, Eurosurveillance, or the web pages of the World Health Organization (WHO). Using software programs, the HTML or RSS/XML of a web page 400 can be parsed, screen scraped, or text scanned for one or more information units 401, 402, 403 and sub-units 404, 405. The information unit and sub-units can be extract titles, URL, date, info text for an alert. The information units scraped or scanned may be compared manually or automatically with a look up searchable database, table or the like.

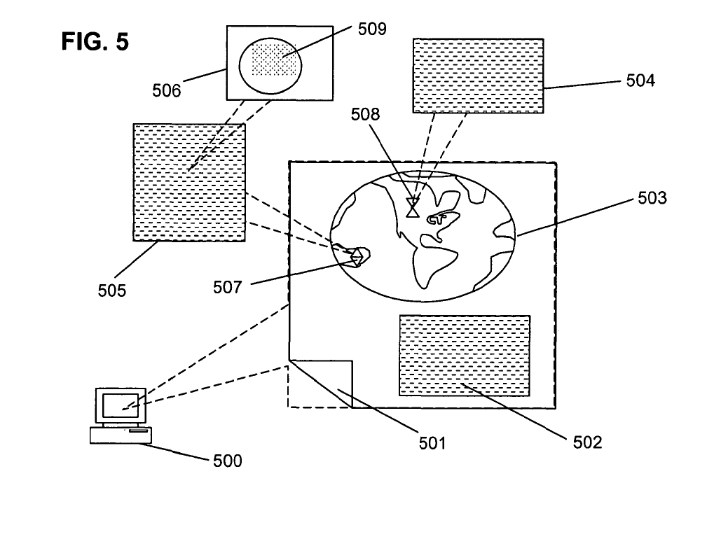

FIG. 5 is another illustration of the use for the invention information generated and presented to a user on a computing terminal. A computing terminal 500 can be used to access a computing server 116, 200 to observe various diagnostic related events using contextual diagnostic information 115 and addition information from the World Wide Web. A web page 501 can contain one or more text information 502 and one or more graphical information 503. The graphical information 503 can be information obtained and stored in one or more databases 117, 118, 211, 307, 308, 309, 406, 407, 408 described by the invention. One example is a satellite image of the earth with additional information or point of interests presented with the satellite image. For example, a point of interest can be an HTML linked 507 specific location having a network of one or more diagnostic device 100, 201, 206. A point of interest can be HTML linked specific location 508 with news content obtained from a scraping data collection method and presented on a web page 501. A user can activate an HTML link 507 by clicking on (selecting) the link to obtain additional linked information 505. The linked information 505 can contain, for example, a cluster analysis result 509 of the number of cases 506 of patients with flu symptoms located at specific emergency rooms in specific cities. The HTML link 508 can contain for example, news 508 relating to an out break of SARS. The web-page and one or more HTML link presents the highest level of integration of diagnostic information collected in a distributed or centralized system comprising diagnostic devices 100, 201, 206 and the World Wide Web to a user. The computer terminal 500 can be implemented in alternative forms of Internet access appliances and various other consumer products through which information retrieval and display may be conducted including a cellular phone, personal digital assistance, wireless computers (palm-based, wearable, mobile phones, etc.).

Various user interface features, screens, and/or web pages made available at a terminal 500 can be associated with embodiments of the present invention. The screens or web pages may be implemented in C++, Java, or JavaScript, or as web pages under XML (Extensible Markup Language), HTML (Hypertext Markup Language), Flash! ASP.net, or any other scripts/methods of creating displayable data, such as the Wireless Access Protocol (“WAP”). The screens or web pages provide facilities to receive input data, such as a form with fields to be filled in, pull-down menus or entries allowing one or more of several options to be selected, buttons, sliders, hypertext links or other known user interface tools for receiving user input. While certain ways of displaying information to users is shown and described with respect to certain figures, those skilled in the relevant art will recognize that various other alternatives may be employed.

In summary, it is believed that the present process provide the highest level of integration and dissemination of diagnostic information collected in a distributed or centralized system comprising diagnostic devices and the Internet (global computing network). The information can be monetized through at least one transaction fee for providing products and/or mediating services with one or more business models incorporating one or more processes of the present invention. The system may be implemented in a more limited fashion, such as to integrate only certain types of resources or for the purposes of data acquisition and analysis alone. However, in any situation, the system may be further expanded by the inclusion of software, firmware, or hardware modules, or by the coupling of additional or different diagnostic instruments and modalities along with their correlation to other data sources in the analyses performed by the processing system. The resulting system, in conjunction with existing and even future sources of medical data, health related and non-health information provides a compliment and extremely useful communication environment or Web 2.0 community for consumers including patients, clinicians, health care providers, healthcare purchasers, public health officials, and policy makers.

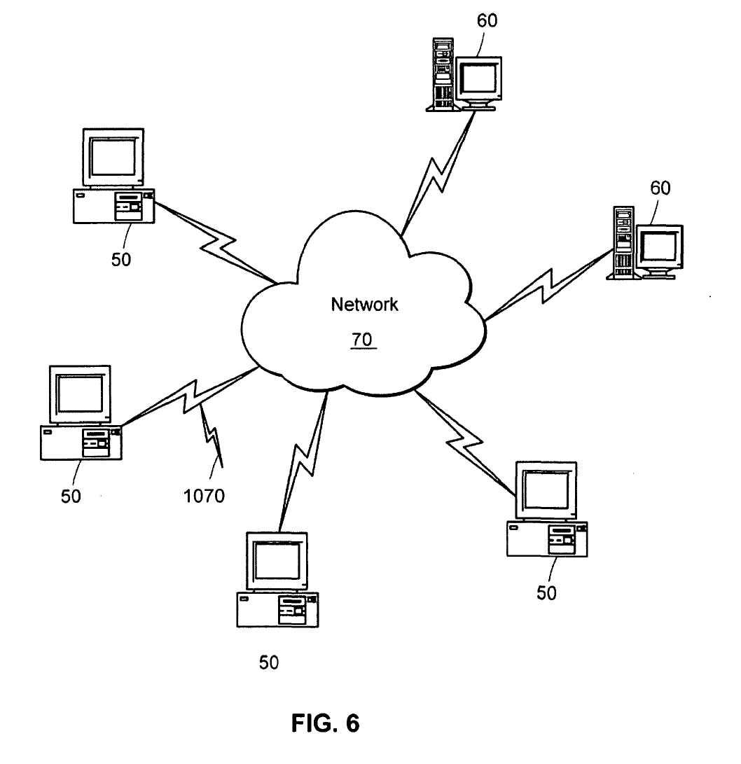

FIG. 6 illustrates a computer network or similar digital processing environment in which embodiments of the present invention may be implemented.

Client computer(s)/devices 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), a worldwide collection of computers, Local area or Wide area networks, and gateways that currently use respective protocols (TCP/IP, Bluetooth, etc.) to communicate with one another. Other electronic device/computer network architectures are suitable.

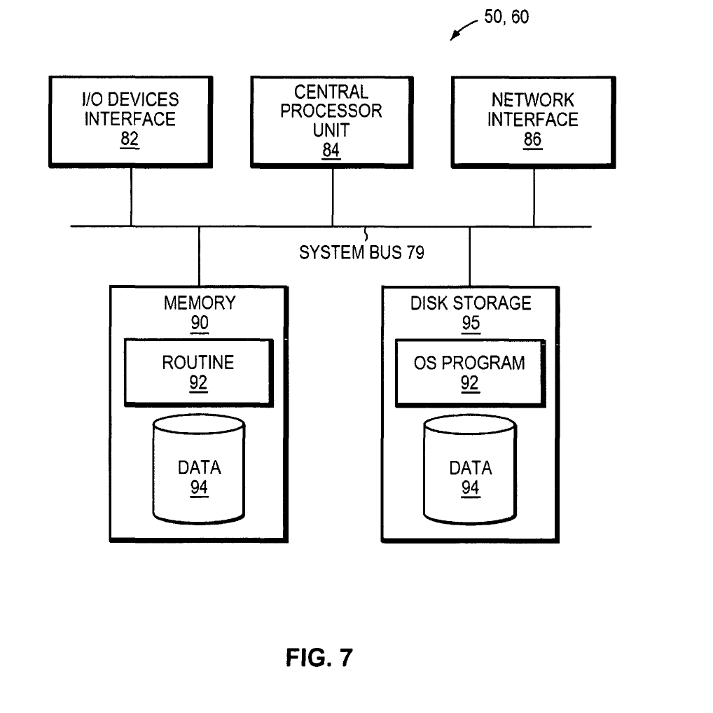

FIG. 7 is a diagram of the internal structure of a computer (e.g., client processor/device 50 or server computers 60) in the computer system of FIG. 6. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 6). Memory 90 provides volatile storage for computer software instructions 92 and data 94 used to implement an embodiment of the present invention (e.g., diagnostic device 100, 201, 206 generated analytical results, augmentation parameters, code for combining the two to form contextual diagnostic information 115 and code 63 for supporting fee for contextual diagnostic information transactions detailed above and below). Disk storage 95 provides non-volatile storage for computer software instructions 92 and data 94 used to implement an embodiment of the present invention. Central processor unit 84 is also attached to system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product 1070 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

A further embodiment of the present invention is a diagnostic method as follows. The diagnostic method includes providing a diagnostic device configured to provide near real time analytical results, testing with the diagnostic device in a testing location, upon use of the diagnostic device, adding augmentation parameters to analytical results generated by the diagnostic device, the augmentation parameters providing context for the analytical results, said adding resulting in contextual diagnostic information; and through a transaction, and enabling access, through a transaction, to the resulting contextual diagnostic information by a user in an access location and/or having the user access, through a transaction, the contextual diagnostic information in the access location.

Any location that is considered to provide diagnostically useful information is a suitable location for testing with the medical diagnostic device. More specifically, locations include, but are not limited to a stationary point of care setting, a field location, within an ambulatory/motional vehicle, or within a body (i.e., with an implanted medical diagnostic device). The medical diagnostic device may be portable but can also be stationary and/or installed or built into/combined with another device. For example, medical diagnostic devices can be combined with a urinal such that the sample collection container (and/or medical diagnostic device) is affixed or built into the urinal allowing urine probes to be tested and monitored, typically, automatically and without the need for control by the test subject.

Access to the resulting contextual information can be enabled to a user, through a transaction, on the medical diagnostic device itself using an input/output device of the device. Alternatively, access to the resulting contextual information can be enabled to a user, through a transaction, and through a distributed processing network of which the diagnostic device is a part.

Any location that allows enabling of access to the resulting contextual diagnostic information is a possible access location. The access location can be the testing location, for example, a person self-testing and accessing the resulting contextual diagnostic information at home, or, testing at point of care and access by a care provider at point of care, for example, a physician's office. Alternatively, the testing location can be different from the access location. For example, a person self-testing at home and accessing the resulting contextual diagnostic information in his car, or a person self-testing at home and a care provider of the person accessing the contextual diagnostic information, for example, in a physician's office.

An “analyte of public health concern” as used herein, refers to an analyte that can be tested for with the medical diagnostic device and indicates a concern for public health. For example, analytes can be and/or be indicative of a virus, bacterium, or prion that is considered a public health concern by a public health entity.

An “analyte of airport safety concern” as used herein, refers to an analyte that can be tested for with the medical diagnostic device and its concentration above an allowed concentration indicates a concern for airport safety. For example, analytes of airport safety concern are, but are not limited to analytes of public health concern, analytes indicative of toxins, analytes indicative of explosives and the like. An “analyte of water safety concern” as used herein, refers to an analyte that can be tested for with the medical diagnostic device and its concentration above an allowed concentration indicates a concern for water safety. For example, analytes of water safety concern are but are not limited to analytes indicative of toxins, microorganisms, disinfectants, disinfection, byproducts, inorganic chemicals, organic chemicals, radionuclides and the like. Microorganisms include, for example, Cryptosporidium, Giardia lamblia, Legionella, and viruses. Disinfection byproducts include, for example, bromate, chlorite, haloacetic acids, and trihalomethanes. Disinfectants include, for example, chloramines, chlorine, and chlorine dioxide. Inorganic chemicals include, for example, antimony, arsenic, asbestos, barium, beryllium, cadmium, chromium, copper, cyanide, fluoride, mercury, nitrate, nitrite, selenium and thallium. Organic chemicals include, for example, acrylamide, alachlor, atrazine, benzene, benzo(a)pyrene, carbofuran, carbon tetrachloride, chlordane, chlorobenzene, dalapon, 1,2-dibromo-3-chloropropane (DBCP), o-dichlorobenzene, p-dichlorobenzene, 1,2-dichloroethane, 1,1-dichloroethylene, cis-1,2-dichloroethylene, trans-1,2-dichloroethylene, dichloromethane, 1,2-dichloropropane, di(2-ethylhexyl) adipate, di(2-ethylhexyl) phthalate, dinoseb, dioxin (2,3,7,8-TCDD), diquat, endothall, endrin, epichlorohydrin, ethylbenzene, ethylene dibromide, glyphosate, heptachlor, heptachlor epoxide, hexachlorobenzene, hexachlorocyclopentadiene, lindane, methoxychlor, oxamyl, polychlorinated biphenyls (PCBs), pentachlorophenol zero, picloram, simazine, styrene, tetrachloroethylene, toluene, toxaphene, 2,4,5-TP (Silvex), 1,2,4-trichlorobenzene, 1,1,1-trichloroethane, 1,1,2-trichloroethane, trichloroethylene, vinyl chloride, and xylenes. Radionuclides include, for example, alpha particles, beta particles and photon emitters, radium 226 and radium 228, and uranium.

The medical diagnostic methods of the present invention are based on diagnostic devices configured to provide near real time analytical results. Further, generally, adding augmentation parameters can be performed rapidly so that contextual diagnostic information is also accessible in near real time. For example, for patient care the availability of near real time contextual diagnostic information may allow for near real time medical intervention, for example, drug administration. Near real time availability of contextual diagnostic information from a plurality of medical diagnostic devices that are part of a distributed processing network, allows monitoring and pre-monitoring (i.e., surveillance), through data analysis, for possible correlations between a plurality of contextual diagnostic information obtained form a plurality of medical diagnostic devices. This allows for near real time initiation of medical intervention and/or non-medical responses.

“Augmentation parameters” as used herein, refers to information that provides context for an analytical result obtained through testing with a medical diagnostic device. Suitable augmentation parameters include, but are not limited to, testing parameters such as type of measurement (e.g., type of nuclear magnetic relaxation parameter measured such as T1 and T2), type of analyte measured, data format codifiers and the like, device status parameters such as model number, maintenance parameters (e.g. number of usable consumables such as nanosensor cartridges), geographic location of the diagnostic device, velocity of the diagnostic device (if moving, e.g., in a motional vehicle such as car, space craft etc.), external conditions in the testing location such as temperature, humidity, wind speed etc., analyte information, test subject information, financial information such as monetary value of analytical result, insurance information such as health insurance information of a test subject being tested, medical information of a test subject being tested such as contact information of care provider of test subject, EMT identifier, hospital information, reason for testing (e.g., routine testing, monitoring, emergency event such as car crash, heart attack, etc.), and the like.

Augmentation parameters may be pre-loaded and/or stored on the medical diagnostic device, manually entered (e.g., by a test subject), measured, or obtained from a computing server or personal data device such as wireless handheld devices (e.g., blackberry), identification devices (e.g., electronic passport, electronic drivers license and the like), entertainment devices (e.g., music/video players, portable game devices) and the like. For example, the diagnostic device 100 can communicate with computing server 116 (such as at 120), either manually or automatically, to obtain augmentation parameters 113,114 such as frame-of-reference data, contained in one or more databases 117,118, append the additional information to the test result 112 (steps 121,123) and transmit the combined information 115 back to a computing server 116 (steps 122,124), and store the result in another database 119.

A further embodiment is a medical diagnostic method that includes, providing a plurality of diagnostic devices, each device being configured to provide near real time analytical results and being located in a different test location, testing with the diagnostic devices, upon use of each diagnostic device, adding augmentation parameters to analytical results generated by each diagnostic device, the augmentation parameters providing context for the analytical results, said adding resulting in contextual diagnostic information; and enabling access, through a transaction, to the resulting contextual diagnostic information of each device by a user in an access location and/or having the user access, through a transaction, the contextual diagnostic information of each device in the access location. Preferably, enabling access to the resulting contextual diagnostic information occurs in near-real time relative to the testing. The user can be a person, a single entity or a plurality of entities. The user can access the contextual diagnostic information from each of the devices and use methods known in the art to model, calculate, estimate, and/or map current and future geographical distribution and frequency of analytical results. This can allow, for example, monitoring and modeling progression of epidemics and pandemics of infectious diseases.

A further embodiment of the present invention is a diagnostic method as follows. The diagnostic method includes providing a diagnostic device configured to provide near real time analytical results, testing with the diagnostic device in a testing location, upon use of the diagnostic device, adding augmentation parameters to analytical results generated by the diagnostic device, the augmentation parameters providing context for the analytical results, said adding resulting in contextual diagnostic information; and through a transaction, and enabling access, through a transaction, to the resulting contextual diagnostic information by a user in an access location and/or having the user access, through a transaction, the contextual diagnostic information in the access location, wherein accessing the contextual diagnostic information comprises through an advertising transaction, blocking and/or receiving advertising information adapted to the contextual diagnostic information.

User of the present methods can access the resulting contextual diagnostic information through a transaction. The transaction can be, for example, financed through a database subscription or by a fee for use. Further, user can request and/or block, through an advertising transaction, advertising information that is provided upon and during accessing of the contextual diagnostic information. The advertising transaction can be, for example financed through a database subscription or by a fee for use. Typically, the user may request advertising information to be provided concurrently or linked to the contextual diagnostic information that provides a benefit to the user and may block advertising information that does not provide a benefit.

Further, generally, the advertising information is adapted to the contextual diagnostic information. For example, if the contextual diagnostic information allows for diagnosis information and the advertising information is selected in view of the diagnosis information, and/or the advertising information is selected in view of an analyte being tested with the diagnostic device.

Advertising information includes, but is not limited to the following advertising types: new drug information, existing drug information, care provider information, hospital or clinical care setting information, treatment information, diagnosis information, diagnostic device accessories information, diagnostic device consumables information, diagnostic device maintenance information, care provider appointment scheduling information, medical help information, or non- diagnostic/non-medical information.

For example, a person self-testing for an analyte indicative of a specific disease, may request advertising information such as an explanation of the analytical result in layman terms, links to further information regarding the disease, information regarding care providers in the area specializing in the treatment of the disease, information treatment options of the disease, treatment options, health care provider information (e.g., indicating coverage of treatment options, need for referral, deductible, co-pay etc.). Further, the person may block advertising information, for example, advertising for alternative medicine treatments, local pharmacy advertising, requests for applying as participant in clinical trials and patient surveys.

The status of a medical diagnostic device, for example, the number of medical diagnostic device consumables present at the point of care location, may be monitored by the medical diagnostic device and/or part of the distributed processing network, and as an augmentation parameter added to the analytical result to result in contextual diagnostic information. Based on the status of the medical diagnostic device, for example, if an NMR based medical diagnostic device using nanosensor cartridges as consumables is running low on these cartridges, diagnostic device consumables information may appear upon and/or during accessing the contextual diagnostic information, informing the user, for example, to approve purchase and sending of new cartridges to the testing location.

A further embodiment of the present invention is a diagnostic method as follows. The diagnostic method includes providing a diagnostic device configured to provide near real time analytical results, testing with the diagnostic device in a testing location, upon use of the diagnostic device, adding augmentation parameters to analytical results generated by the diagnostic device, the augmentation parameters providing context for the analytical results, said adding resulting in contextual diagnostic information; and through a transaction, and enabling access, through a transaction, to the resulting contextual diagnostic information by a user in an access location and/or having the user access, through a transaction, the contextual diagnostic information in the access location, wherein testing is controlled remotely by the user, the access location being remote from the testing location.

Medical diagnostic devices may test automatically, that is, without the need for external control, or with partial external control, for example, a user may initiate automatic testing at the testing location or remotely. This is generally preferred for standard monitoring or surveillance of analytes using standard protocols that may be included in the medical diagnostic device or communicated to the medical diagnostic device without user involvement. However, for some applications user control over the testing with the medical diagnostic device may be desired.

A further embodiment of the present invention is a diagnostic method as follows. The diagnostic method includes providing a diagnostic device configured to provide near real time analytical results, testing with the diagnostic device in a testing location, upon use of the diagnostic device, adding augmentation parameters to analytical results generated by the diagnostic device, the augmentation parameters providing context for the analytical results, said adding resulting in contextual diagnostic information; and through a transaction, and enabling access, through a transaction, to the resulting contextual diagnostic information by a user in an access location and/or having the user access, through a transaction, the contextual diagnostic information in the access location, wherein the testing location is a drive-through diagnostic center configured to allow testing with the diagnostic medical device of a person within a vehicle.

A “drive-through medical diagnostic center” as used herein, refers to a stationary medical diagnostic center adapted to allow customers to drive their vehicle into the center and be tested with the medical diagnostic device without having to leave the vehicle.

Medical diagnostic devices that can be used in the diagnostic methods and corresponding medical diagnostic systems of the present invention, are devices that allow real-time or near real-time determination/testing of the presence and/or concentration of analytes and/or real-time or near real-time determination/testing of sample conditions, for example, coagulation state of a blood sample. Suitable medical diagnostic devices and testing methods using the medical diagnostic devices of the present invention are described in the following patent applications, which are herewith incorporated by reference in their entirety: U.S. Provisional Application No. 60/952,143, filed Jul. 26, 2007 by Thomas Jay Lowery, Jr. et al; U.S. application Ser. No. 11/513,503, filed Aug. 31, 2006 by W. David Lee (now U.S. Pat. No. 7,564,245, issued Jul. 1, 2009); U.S. Provisional Application No. 60/857,742, filed Nov. 8, 2006 by W. David Lee et al (now Ser. No. 12/514,250); U.S. Provisional Application No. 60/904,685, filed Mar. 2, 2007 by Jim Koziarz et al; U.S. Provisional Application No. 61/063,389, filed Feb. 1, 2008 by James J. Koziarz et al; U.S. Provisional Application No. 60/919,236, filed Mar. 21, 2007 by Doug Levinson et al; U.S. Provisional Application No. 61/063,422, filed Feb. 1, 2008 by Douglas A. Levinson et al; U.S. Provisional Application No. 60/915,797, filed May 3, 2007 by Tom Lowery et al; U.S. Provisional Application No. 60/912,298, filed Apr. 17, 2007 by Tom Lowery et al; U.S. Provisional Application No. 61/066,504, filed Feb. 21, 2008 by Thomas J. Lowery, Jr. et al; U.S. Provisional Application No. 60/937,067, filed Jun. 25, 2007 by Thomas J. Lowery, Jr. et al; U.S. Provisional Application No. 60/995,830, filed Sep. 28, 2007 by Thomas Jay Lowery, Jr. et al; U.S. Provisional Application No. 60/995,724, filed Sep. 28, 2007 by Thomas Jay Lowery, Jr. et al (now PCT/US2008/011240, filed Sep. 29, 2008); U.S. Provisional Application No. 60/995,758, filed Sep. 28, 2007 by Sonia Taktak et al; U.S. Provisional Application No. 61/002,021, filed Nov. 6, 2007 by Thomas J. Lowery, Jr.; U.S. Provisional Application No. 61/000,519, filed Oct. 26, 2007 by Sonia Taktak; U.S. Provisional Application No. 61/002,022, filed Nov. 6, 2007 by Pablo J. Prado et al; U.S. Provisional Application No. 61/008,991, filed Dec. 21, 2007 by Pablo J. Prado et al (now PCT/US2008/012592 filed Nov. 6, 2008); U.S. Provisional Application No. 61/008,646, filed Dec. 21, 2007 by Thomas J. Lowery, Jr. et al (now PCT/US2008/013911 filed Dec. 19, 2008); U.S. Provisional Application No. 61/008,669, filed Dec. 21, 2007 by Thomas J. Lowery, Jr. et al; U.S. Provisional Application entitled “Integrated Magnetic Resonance Apparatus and Single-Step Particle-Based Diagnostics by Means of a Switchable Magnetic Field” (Attorney Docket Number: 4203.1019-000), filed Mar. 4, 2008 by Pablo J. Prado et al, U.S. patent application Ser. No. 10/165,258, filed Jun. 6, 2002, U.S. patent application Ser. No. 11/431,247, filed May 9, 2006 (now Ser. No. 12/612,126), U.S. Provisional Patent Application No. 61/127,514, filed May 14, 2008, U.S. Provisional Patent Application No. 61/068,211, filed Mar. 5, 2008, U.S. Provisional Application No.: (Not yet assigned), filed Jul. 11, 2008 by Thomas J. Lowery, Jr. et al., entitled: Methods for Directed Assay Design Based on Analytical Method Guidance (61/134,668), and U.S. Provisional Application No.: (Not yet assigned), filed Jul. 11, 2008 by Thomas J. Lowery, Jr. et al., entitled: Agglomerative MRSw Assays Operating in the ‘Increasing T2’ Regime and Dispersive MRSw Assays Operating in the ‘Decreasing T2’ Regime (61/134,606).

Typically, the medical diagnostic device is a nuclear magnetic resonance device, for example, a magnetic resonance relaxometer; and, typically, testing with the diagnostic device to obtain analytical results includes measuring/determining a nuclear magnetic resonance parameter, more typically, T₂.

Further embodiments of the present invention are medical diagnostic systems that allow performing the medical diagnostic methods described herein.

It is contemplated that devices, systems, methods, and processes of the claimed invention encompass variations and adaptations developed using information from the embodiments described herein. Adaptation and/or modification of the devices, systems, methods, and processes described herein may be performed by those of ordinary skill in the relevant art.

Throughout the description, where devices and systems are described as having, including, or comprising specific components, or where processes and methods are described as having, including, or comprising specific steps, it is contemplated that, additionally, there are devices and systems of the present invention that consist essentially of, or consist of, the recited components, and that there are processes and methods according to the present invention that consist essentially of, or consist of, the recited processing steps.

It should be understood that the order of steps or order for performing certain actions is immaterial so long as the invention remains operable. Moreover, two or more steps or actions may be conducted simultaneously.

The mention of the Internet, web pages thereon and web browsers is but one example of global computer network environments in which the present invention may be implemented. Other global computer network formats and configurations are suitable.

The mention of real-time aspects of the present invention are intended to include near real-time and effectively similar time frames and are not limited to exacting moments of time.

The mention herein of any publication, for example, in the Background section, is not an admission that the publication serves as prior art with respect to any of the claims presented herein. The background section is presented for purposes of clarity and is not meant as a description of prior art with respect to any claim.

### Equivalents

While the invention has been particularly shown and described with reference to specific preferred embodiments or examples, it should be understood by those skilled in the art that various changes in form and detail may be made therein without departing from the spirit and scope of the invention as defined by the appended claims.

1-77. (canceled)

78. A medical diagnostic system comprising:
a diagnostic device configured to provide near real time analytical results; and
a processor member, responsive to the diagnostic device, configured to add augmentation parameters to the analytical results to form contextual diagnostic information,
wherein user access to the contextual diagnostic information is provided through a fee for information transaction.

79. The medical diagnostic system of claim 78, wherein the diagnostic device comprises a device configured to detect at least one of an analyte and a biomarker, the device having a support defining a well for holding a liquid sample comprising magnetic particles and the analyte or biomarker, the magnetic particles having binding moieties linked thereto, and an RF coil configured to detect a magnetic resonance response produced by exposing the liquid sample to a bias magnetic field created using one or more magnets and an RF excitation.

80. The medical diagnostic system of claim 79, wherein the diagnostic device is at least one of portable and part of a distributed processing network.

81. The medical diagnostic system of claim 79, wherein the augmentation parameters include any one or combination of: position of the diagnostic device, location of the diagnostic device, velocity of the diagnostic device and frame of reference data.

82. The medical diagnostic system of claim 79, wherein the transaction comprises at least one of a database subscription and a fee for use at point of care communication.

83. The medical diagnostic system of claim 79, wherein the analyte is selected from the group consisting of a protein, a nucleic acid, a cell, a carbohydrate, a therapeutic agent, a polymer, and a virus.

84. The medical diagnostic system of claim 79, wherein the diagnostic device is in a testing location and the fee for information transaction is in an access location.

85. The medical diagnostic system of claim 84, wherein the diagnostic device is implanted, portable and/or part of a distributed processing network.

86. The medical diagnostic system of claim 84, wherein the testing location is different from the access location.

87. The medical diagnostic system of claim 84, wherein the testing location is a stationary point of care setting, a field location, or within an ambulatory/motional vehicle.

88. The medical diagnostic system of claim 84, wherein the testing location is a drive-through diagnostic center, and the diagnostic medical device is configured to allow testing with the diagnostic medical device of a person within a vehicle.

89. A medical diagnostic method comprising:
providing near real time analytical results using a diagnostic device;
adding augmentation parameters to the analytical results to form contextual diagnostic information using a processor member responsive to the diagnostic device; and
providing user access to the contextual diagnostic information through a fee for information transaction.

90. The medical diagnostic method of claim 89, wherein the providing near real time analytical results comprises using a device configured to detect at least one of an analyte and a biomarker, the device having a support defining a well for holding a liquid sample comprising magnetic particles and the analyte or biomarker, the magnetic particles having binding moieties linked thereto, and an RF coil configured to detect a magnetic resonance response produced by exposing the liquid sample to a bias magnetic field created using one or more magnets and an RF excitation.

91. The medical diagnostic method of claim 90, wherein the using a device comprises using at least one of portable device and part of a distributed processing network.

92. The medical diagnostic method of claim 90, further comprising using augmentation parameters including any one or combination of: position of the diagnostic device, location of the diagnostic device, velocity of the diagnostic device and frame of reference data.

93. The medical diagnostic method of claim 90, wherein the transaction comprises at least one of a database subscription and a fee for use at point of care communication.

94. The medical diagnostic method of claim 90, wherein the analyte is selected from the group consisting of a protein, a nucleic acid, a cell, a carbohydrate, a therapeutic agent, a polymer, and a virus.

95. The medical diagnostic method of claim 90, wherein the diagnostic device is implanted, portable and/or part of a distributed processing network.

96. The medical diagnostic method of claim 95, further comprising using the diagnostic device in a testing location and performing the fee for information transaction in an access location.

97. The medical diagnostic method of claim 95, wherein the testing location is different from the access location.

98. The medical diagnostic method of claim 95, wherein the testing location is a stationary point of care setting, a field location, or within an ambulatory/motional vehicle.

99. The medical diagnostic method of claim 95, wherein the testing location is a drive-through diagnostic center, and the diagnostic medical device is configured to allow testing with the diagnostic medical device of a person within a vehicle.
